(19) United States
(12) Patent Application Publication
(10) Pub. No.: US 2026/0315153 A1
(43) Pub. Date: Oct. 15, 2026

(54) SYSTEMS AND METHODS OF DETECTING AND CORRECTING FOR PATIENT AND/OR IMAGING SYSTEM MOVEMENT FOR TARGET OVERLAY

(71) Applicant: Covidien LP, Mansfield, MA (US)

(72) Inventors: Irina SHEVLEV, Ashdod (IL); Guy ALEXANDRONI, Yehud-Monosson (IL); Ariel BIRENBAUM, Raanana (IL)

(21) Appl. No.: 19/116,485

(22) PCT Filed: Oct. 10, 2023
(86) PCT No.: PCT/IB2023/060172

§ 371 Date: Mar. 28, 2025

Related U.S. Application Data
(60) Provisional application No. 63/416,334, filed on Oct. 14, 2022.
(60) Provisional application No. 63/429,147, filed on Dec. 1, 2022.

(51) Int. Cl. A61B 34/20; A61B 90/00

(52) U.S. Cl. CPC A61B 34/20; A61B 90/39; A61B 2034/2051; A61B 2090/376

(57) ABSTRACT

Systems and methods for visualizing navigation of a medical device relative to a target use live fluoroscopic video and update a position of a target overlaid on the live fluoroscopic video. The systems and methods involve updating the 2D position of the target overlay based on patient movement sensed by patient reference sensors. In aspects, the medical device tip may be automatically detected, thus eliminating the need for a reference frame. The setup procedure is automatically performed in response to detecting movement of the fluoroscopic imaging system. The pose estimation may be optimized by using a non-periodic grid. The systems and methods may incorporate digital zoom that may pan a fluoroscopic image to focus on the target. The systems and methods may involve tracking which part of the target was covered by previous procedure iterations.

### CROSS-REFERENCE TO RELATED APPLICATIONS

This application claims priority to and the benefit of U.S. Application Nos. 63/416,334, filed Oct. 14, 2022 and 63/429,147, filed Dec. 1, 2022.

### FIELD

This disclosure relates to the field of visualizing the navigation of medical devices, such as biopsy or ablation tools, relative to targets, confirming the relative positions, and monitoring and compensating for movement of the patient and/or the live imaging system.

### BACKGROUND

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may be needed to navigate the medical device to the target in a safe and accurate manner (e.g., without causing damage to other organs or tissue).

For example, an endoscopic approach has proven useful in navigating to areas of interest within a patient, and particularly so for areas within luminal networks of the body such as the lungs. To enable the endoscopic approach, and more particularly the bronchoscopic approach in the lungs, endobronchial navigation systems have been developed that use previously acquired MRI data or CT image data to generate a three-dimensional (3D) rendering, model, or volume of the particular body part such as the lungs.

The resulting volume generated from the MRI scan or CT scan is then utilized to create a navigation plan to facilitate the advancement of a navigation catheter (or other suitable medical device) through a bronchoscope and a branch of the bronchus of a patient to an area of interest. A locating or tracking system, such as an electromagnetic (EM) tracking system, may be utilized in conjunction with, for example, CT data, to facilitate guidance of the navigation catheter through the branch of the bronchus to the area of interest. In certain instances, the navigation catheter may be positioned within one of the airways of the branched luminal networks adjacent to, or within, the area of interest to provide access for one or more medical instruments.

However, a 3D volume of a patient's lungs, generated from previously acquired scans, such as CT scans, may not provide a basis sufficient for accurate guiding of medical devices or instruments to a target during a navigation procedure. In some cases, the inaccuracy is caused by deformation of the patient's lungs during the procedure relative to the lungs at the time of the acquisition of the previously acquired CT data. This deformation (CT-to-Body divergence) may be caused by many different factors including, for example, changes in the body when transitioning from between a sedated state and a non-sedated state, the bronchoscope changing the patient's pose, the bronchoscope pushing the tissue, different lung volumes (e.g., the CT scans are acquired during inhale while navigation is performed during breathing), different beds, different days, etc.

Thus, another imaging modality is needed to visualize medical devices and targets in real-time and enhance the in-vivo navigation procedure. Furthermore, to navigate medical devices accurately and safely to a remote target, for example, for biopsy or treatment, both the medical device and the target should be visible in a guidance system.

### SUMMARY

The techniques of this disclosure generally relate to systems and methods for visualizing navigation of a medical device relative to a target using live fluoroscopic video, repeating a setup procedure in response to detecting movement of a fluoroscopic imaging system, and updating a position of the target overlaid on the live fluoroscopic video in response to detecting movement of a patient.

In one aspect, the disclosure provides a method. The method includes performing a setup procedure including determining a first position of a tip of a catheter in a reference frame of the live fluoroscopic video and determining a position of the fluoroscopic imaging system based on the first position of the tip of the catheter in the reference frame. The method also includes receiving live fluoroscopic video from the fluoroscopic imaging system, and displaying the live fluoroscopic video. The method also includes projecting three-dimensional (3D) coordinates from an electromagnetic (EM) sensor disposed at the tip of the catheter onto the live fluoroscopic video based on the position of the fluoroscopic imaging system, which yields projected 3D coordinates, overlaying a target on the live fluoroscopic video. The method also includes determining that a second position of the tip of the catheter in the live fluoroscopic video is not at or near the projected 3D coordinates, and in response to determining that the second position of the tip of the catheter in the live fluoroscopic video is not at or near the projected 3D coordinates, determining that the fluoroscopic imaging system has moved and repeating performing the setup procedure. The method also includes receiving a position of a patient from one or more second EM sensors disposed on the patient and determining that the patient has moved based on the position of the patient. The method also includes in response to determining that the patient has moved, determining an updated position of the target, and overlaying the target on the live fluoroscopic video at the updated position.

Implementations of the method may also include one or more of the following features. The method may include determining that the catheter is not aligned with the target and, in response to determining that the catheter is not aligned with the target, updating an appearance of the target overlaying the live fluoroscopic video. Updating the appearance of the target may include changing a color of the target, highlighting the target, or applying a line pattern to the target. The method may include panning a view of the live fluoroscopic video such that the target is at a center of the view of the live fluoroscopic video. Determining that the patient has moved may include determining a reference position of the one or more second EM sensors, and determining that the distance between the position of the patient and the reference position is greater than a threshold distance.

The method may include determining possible translations of the fluoroscopic imaging system based on a periodic grid appearing in the live fluoroscopic video; selecting, from the possible translations, a translation for which the projected 3D coordinates most closely matches the second position of the tip of the catheter in the live fluoroscopic video, yielding a selected translation; and determining the position of the fluoroscopic imaging system based on the first position of the tip of the catheter in the reference frame and the selected translation. The method may include tracking portions of the target that have been biopsied and displaying the portions of the target that have been biopsied. The fluoroscopic imaging system may be a 3D fluoroscope.

In another aspect, the disclosure provides a system for guiding navigation of a biopsy tool in a patient. The system includes an electromagnetic (EM) field generator that generates an electromagnetic field, a first EM sensor disposed at a tip of a catheter, one or more second EM sensor disposed on the patient, and a display. The system also includes a processor and a memory having stored thereon instructions, wherein when the instructions are executed by the processor, the processor performs a setup procedure including determining a first position of a tip of a catheter in a reference frame of the live fluoroscopic video and determining a position of the fluoroscopic imaging system based on the first position of the tip of the catheter in the reference frame.

When the instructions are executed by the processor, the processor also receives live fluoroscopic video from a fluoroscopic imaging system, displays the live fluoroscopic video, projects three-dimensional (3D) coordinates from an electromagnetic (EM) sensor disposed at the tip of the catheter onto the live fluoroscopic video based on the position of the fluoroscopic imaging system, yielding projected 3D coordinates, overlays a target on the live fluoroscopic video, determines that a second position of the tip of the catheter in the live fluoroscopic video is not at or near the projected 3D coordinates, and in response to determining that the second position of the tip of the catheter in the live fluoroscopic video is not at or near the projected 3D coordinates, determines that the fluoroscopic imaging system has moved and repeats performing the setup procedure.

When the instructions are executed by the processor, the processor also receives a position of a patient from the one or more second EM sensors disposed on the patient, determines that the patient has moved based on the position of the patient, in response to determining that the fluoroscopic imaging system has moved or the patient has moved, determines an updated position of the target, and overlays the target on the live fluoroscopic video at the updated position.

Implementations of the system may also include one or more of the following features. When the instructions are executed by the processor, the processor may calculate a patient coordinate frame of reference based on the EM field sensed by the EM sensors. When the instructions are executed by the processor, the processor may determine that the catheter is not aligned with the target and, in response to determining that the catheter is not aligned with the target, update an appearance of the target overlaying the live fluoroscopic video. Updating the appearance of the target overlaying the live fluoroscopic video may include changing a color of the target, highlighting the target, or applying a line pattern to the target.

In aspects, when the instructions are executed by the processor, the processor may pan a view of the live fluoroscopic video such that the target is at a center of the view of the live fluoroscopic video. When the instructions are executed by the processor, the processor determines possible translations of the fluoroscopic imaging system based on a periodic grid appearing in the live fluoroscopic video; selects, from the possible translations, a translation for which the projected 3D coordinates most closely matches the second position of the tip of the catheter in the live fluoroscopic video, yielding a selected translation; and determines the position of the fluoroscopic imaging system based on the first position of the tip of the catheter in the reference frame and the selected translation. When the instructions are executed by the processor, the processor may track portions of the target that have been biopsied, and display the portions of the target that have been biopsied. The fluoroscopic imaging system may be a 3D fluoroscope.

In still another aspect, the disclosure provides a system. The system includes an electromagnetic (EM) field generator that generates an electromagnetic field, one or more EM sensors disposed on a body of a patient, and a display. The system also includes a processor coupled to the display and a memory coupled to the processor and having stored thereon instructions, which when executed by the processor, cause the processor to display, on the display, a screen including a three-dimensional (3D) view of a 3D model of a target from a perspective of a tip of a medical device, and display, in the screen, a live two-dimensional (2D) fluoroscopic view showing the medical device. The instructions, when executed by the processor, may also cause the processor to overlay a target mark, which corresponds to the 3D model of the target, on the live 2D fluoroscopic view, determine that the body of the patient has moved based on one or more signals received from the one or more EM sensors, and, in response to determining that the body of the patient has moved, update a 2D position of the target mark overlaying the live 2D fluoroscopic view.

Implementations of the system may also include one or more of the following features. Determining that the body of the patient has moved may include determining that an EM sensor of the one or more EM sensors disposed on the patient has moved greater than a threshold amount. Determining that the body of the patient has moved may include determining that a combination of two or more of the EM sensors disposed on the patient have moved greater than a threshold amount in a particular direction.

In still another aspect, the disclosure provides a method. The method includes detecting a nonperiodic grid of markers in at least one fluoroscopic image captured by a fluoroscopic imaging system, yielding a detected nonperiodic grid of markers, and determining a position of the fluoroscopic imaging system based on the detected nonperiodic grid of markers. The method also includes displaying live fluoroscopic video from the fluoroscopic imaging system; projecting three-dimensional (3D) coordinates from an electromagnetic (EM) sensor disposed at the tip of the catheter onto the live fluoroscopic video based on the position of the fluoroscopic imaging system, yielding projected 3D coordinates; and overlaying a target on the live fluoroscopic video. The method also includes determining that a second position of the tip of the catheter in the live fluoroscopic video is not at or near the projected 3D coordinates, and, in response to determining that the second position of the tip of the catheter in the live fluoroscopic video is not at or near the projected 3D coordinates, determining that a fluoroscopic imaging system has moved. The method also includes in response to determining that the fluoroscopic imaging system has moved, repeating the detecting the nonperiodic grid of markers and determining the position of the fluoroscopic imaging system.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects of the disclosure are described hereinbelow with references to the drawings, wherein:

FIG. 1 is a diagram of a system for navigating to targets via luminal networks in accordance with the disclosure;

FIG. 2 is a screen shot of an example of a user interface for confirming local registration in accordance with the disclosure;

FIG. 3 is a screen shot of an example of a navigation user interface with a pop-up message inquiring about whether to continue with a target overlay feature in accordance with the disclosure;

FIG. 4 is a screen shot of an example of a navigation user interface without a target overlay feature in accordance with the disclosure;

FIG. 5 is a screen shot of an example of a navigation user interface with the target overlay feature in accordance with the disclosure;

FIG. 6 is a screen shot of an example of a navigation user interface illustrating a screen that appears when a “Target Overlay” tab is selected in accordance with the disclosure;

FIG. 7 is a flowchart of an example of a method for visualizing a medical procedure on live fluoroscopic video in accordance with the disclosure;

FIG. 8 is a screen shot of an example navigation user interface showing a target marker overlaid on a real-time two-dimensional (2D) fluoroscopic view in accordance with the disclosure;

FIG. 9 is a flowchart that illustrates another method for updating a position of a target overlaying live fluoroscopic video in accordance with the disclosure;

FIG. 10 is a diagram that illustrates a nonperiodic grid of radiopaque markers that may be used in the method of FIG. 9;

FIG. 11 is a flowchart that illustrates a method of tracking and displaying portions of a target that have been biopsied;

FIG. 12 is a flowchart of an example of a method of visualizing the navigation of a medical device relative to a target; and

FIG. 13 is a diagram of a system in accordance with the disclosure for navigating to a target and displaying user interfaces in accordance with the disclosure.

### DETAILED DESCRIPTION

A fluoroscopic imaging system may be used by a clinician, for example, to visualize the navigation of a medical device and confirm the placement of the medical device after it has been navigated to a desired location. However, although fluoroscopic images show highly dense objects, such as metal tools, bones, and large soft-tissue objects, e.g., the heart, the fluoroscopic images may not clearly show small soft-tissue objects of interest, such as lesions. Furthermore, the fluoroscopic images are two-dimensional projections. Therefore, an X-ray volumetric reconstruction is needed to enable identification of soft tissue objects and navigation of medical devices to those objects.

Several solutions exist that provide 3D or volume reconstruction. One solution is a CT machine, which algorithmically combines multiple X-ray projections from known, calibrated X-ray source positions into a volume, in which soft tissues are more visible. For example, a CT machine can be used with iterative scans during a procedure to provide guidance through the body until the tool or tools reach the target. This is a tedious procedure, as it requires several full CT scans, a dedicated CT room, and blind navigation between scans. In addition, each scan requires the staff to leave the room due to high levels of ionizing radiation and exposes the patient to the radiation. Another solution is a cone-beam CT machine. However, the cone-beam CT machine is expensive and, like the CT machine, only provides blind navigation between scans, requires multiple iterations for navigation, and requires the staff to leave the room. In some example aspects, the systems and methods of this disclosure combine the benefits of CT machines and fluoroscopic imaging systems to help clinicians navigate medical devices to targets, including small soft-tissue objects.

In an electromagnetic navigation procedure, planning, registration, and navigation are performed to ensure that a medical device, e.g., a biopsy tool, follows a planned path to reach a target, e.g., a lesion, so that a biopsy or treatment of the target can be completed. Following the navigation phase, fluoroscopic images may be captured and utilized in a local registration process to reduce CT-to-body divergence. After the local registration process, the locatable guide may be removed from the extended working channel and a medical device, e.g., a biopsy tool, is introduced into the extended working channel and navigated to the target to perform the biopsy or treatment of the target, e.g., the lesion.

In navigating the medical device to the target, clinicians may use a live 2D fluoroscopic view to visualize the position of the medical device relative to the target. While the medical device may be visible in the live fluoroscopic view, some targets, e.g., lesions, may not be visible in the live fluoroscopic view. And electromagnetic navigation cannot be used because some medical devices may not include sensors. Moreover, the user interfaces that are used to advance or navigate a medical device towards the target do not provide enough information regarding the medical device relative to the target, including when the medical device is near the target.

This disclosure features a user interface which overlays a 2D target marker or representation, which corresponds to a three-dimensional model of a target identified in a CT scan, on the live 2D fluoroscopic view so that a clinician can visualize the position of the medical device tip relative to the target. Since the live fluoroscopic view with the target overlay is a two-dimensional view and does not necessarily show whether the medical device is above or below the target, the same user interface also includes a three-dimensional, medical device tip view of the 3D model of the target, which enables a clinician to confirm that the medical device is not above or below the target.

The user interface also provides a graphical indication of whether the medical device is aligned with the target in three dimensions. For example, when the medical device is aligned in three-dimensions with the target, the user interface shows the target overlay in a first color, e.g., green. On the other hand, when the medical device is not aligned with the target in three dimensions, the user interface shows the target overlay in a second color different from the first color, e.g., orange or red.

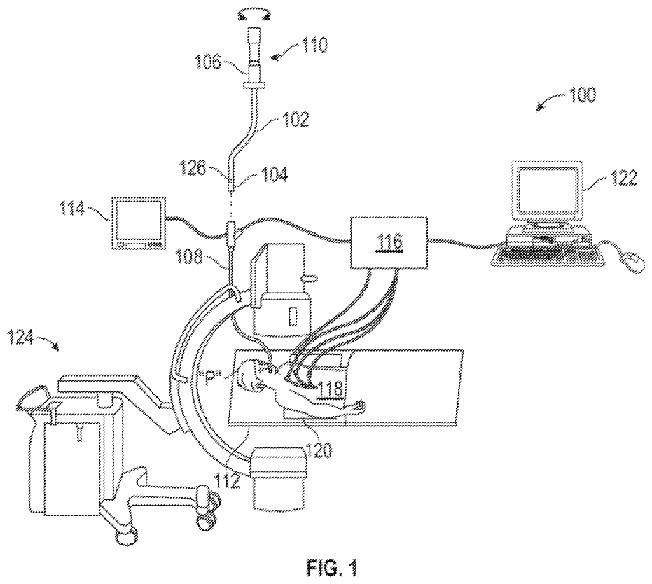

In accordance with aspects of the disclosure, the visualization of intra-body navigation of a medical device, e.g., a biopsy tool, towards a target, e.g., a lesion, may be a portion of a larger workflow of a navigation system, such as an electromagnetic navigation system. FIG. 1 is a perspective view of an exemplary system for facilitating navigation of a medical device, e.g., a biopsy tool, to a soft-tissue target via airways of the lungs. System 100 may be further configured to construct fluoroscopic based three-dimensional volumetric data of the target area from 2D fluoroscopic images. System 100 may be further configured to facilitate approach of a medical device to the target area by using Electromagnetic Navigation Bronchoscopy (ENB) and for determining the location of a medical device with respect to the target.

One aspect of the system 100 is a software component for reviewing of computed tomography (CT) image data that has been acquired separately from system 100. The review of the CT image data allows a user to identify one or more targets, plan a pathway to an identified target (planning phase), navigate a catheter 102 to the target (navigation phase) using a user interface, and confirming placement of a sensor 104 relative to the target. One such EMN system is the ELECTROMAGNETIC NAVIGATION BRONCHOSCOPY® system currently sold by Medtronic PLC. The target may be tissue of interest identified by review of the CT image data during the planning phase. Following navigation, a medical device, such as a biopsy tool or other tool, may be inserted into catheter 102 to obtain a tissue sample from the tissue located at, or proximate to, the target.

As shown in FIG. 1, catheter 102 is part of a catheter guide assembly 106. In practice, catheter 102 is inserted into a bronchoscope 108 for access to a luminal network of the patient P. Specifically, catheter 102 of catheter guide assembly 106 may be inserted into a working channel of bronchoscope 108 for navigation through a patient's luminal network. A locatable guide 110 including a sensor 104 is inserted into catheter 102 and locked into position such that the sensor 104 extends a desired distance beyond the distal tip of catheter 102. The position and orientation of sensor 104 relative to the reference coordinate system, and thus the distal portion of catheter 102, within an electromagnetic field can be derived. The catheter guide assemblies 106 are currently marketed and sold by Medtronic PLC under the brand names SUPERDIMENSION® Procedure Kits, or EDGE™ Procedure Kits, and are contemplated as useable with the disclosure.

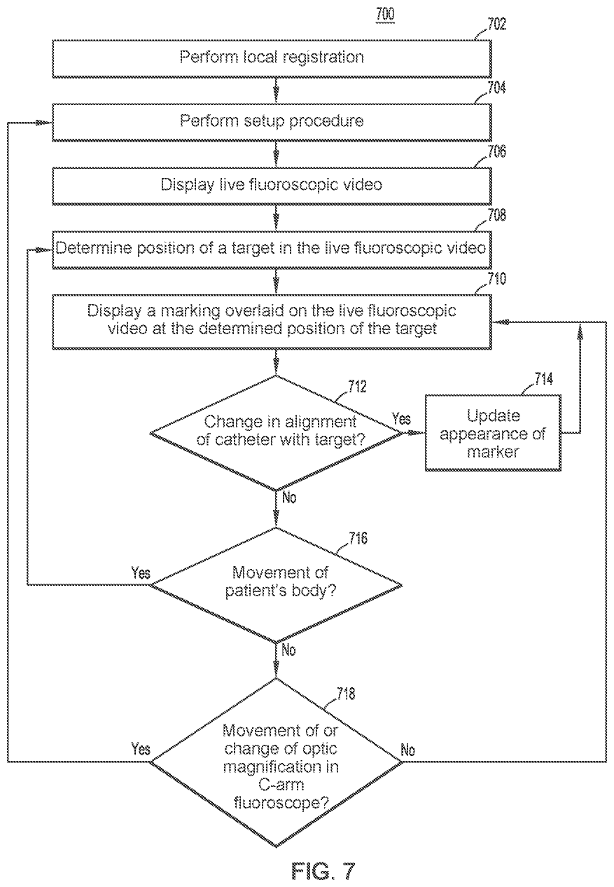

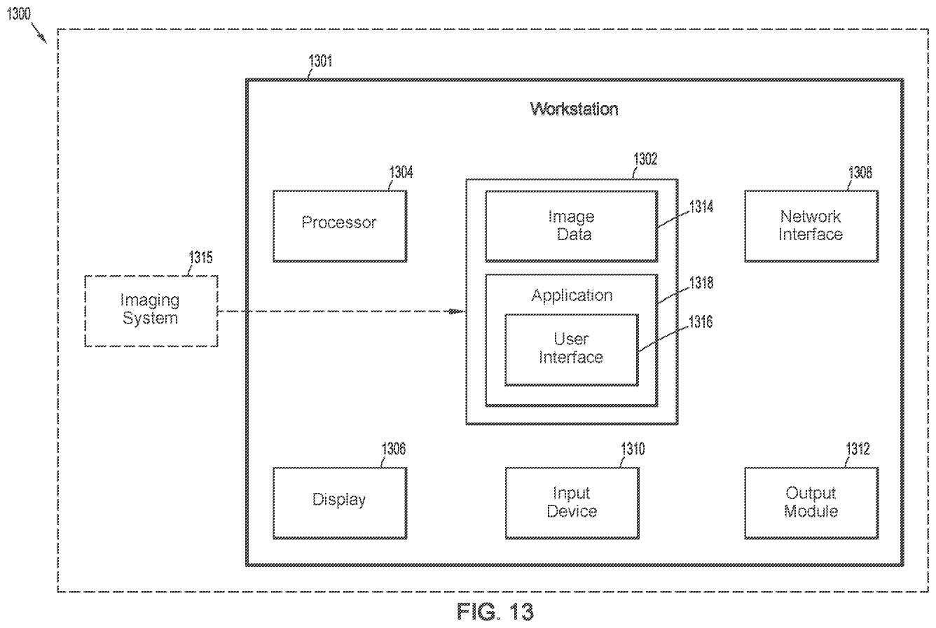

System 100 generally includes an operating table 112 configured to support a patient P, a bronchoscope 108 configured for insertion through patient P's mouth into patient P's airways; monitoring equipment 114 coupled to bronchoscope 108 (e.g., a video display, for displaying the video images received from the video imaging system of bronchoscope 108); a locating or tracking system 116, patient reference sensors 118, and a transmitter mat 120 including a plurality of incorporated markers; and a computer system 122 including software and/or hardware used to facilitate identification of a target, pathway planning to the target, navigation of a medical device to the target, and/or confirmation and/or determination of placement of catheter 102, or a suitable device therethrough, relative to the target. Computer system 122 may be similar to workstation 1501 of FIG. 13 and may be configured to execute the methods of the disclosure including the method of FIGS. 7, 9, 11, and 12.

A fluoroscopic imaging system 124 capable of acquiring fluoroscopic or x-ray images or video of the patient P is also included in this particular aspect of system 100. The images, sequence of images, or video captured by fluoroscopic imaging system 124 may be stored within fluoroscopic imaging system 124 or transmitted to computer system 122 for storage, processing, and display. Additionally, fluoroscopic imaging system 124 may move relative to the patient P so that images may be acquired from different angles or perspectives relative to patient P to create a sequence of fluoroscopic images, such as a fluoroscopic video. The pose of fluoroscopic imaging system 124 relative to patient P and while capturing the images may be estimated via markers incorporated with the transmitter mat 120. The markers are positioned under patient P, between patient P and operating table 112 and between patient P and a radiation source or a sensing unit of fluoroscopic imaging system 124. The markers incorporated with the transmitter mat 120 may be two separate elements which may be coupled in a fixed manner or alternatively may be manufactured as a single unit. Fluoroscopic imaging system 124 may include a single imaging system or more than one imaging system.

Computer system 122 may be any suitable computer system including a processor and storage medium, wherein the processor is capable of executing instructions stored on the storage medium. Computer system 122 may further include a database configured to store patient data, CT data sets including CT images, fluoroscopic data sets including fluoroscopic images and video, fluoroscopic 3D reconstruction, navigation plans, and any other such data. Although not explicitly illustrated, computer system 122 may include inputs, or may otherwise be configured to receive, CT data sets, fluoroscopic images or video and other data described herein. Additionally, the computer system 122 includes a display configured to display graphical user interfaces. The computer system 122 may be connected to one or more networks through which one or more databases may be accessed.

With respect to the planning phase, the computer system 122 utilizes previously acquired CT image data for generating and viewing a three-dimensional model or rendering of patient P's airways, enables the identification of a target on the three-dimensional model (automatically, semi-automatically, or manually), and allows for determining a pathway through patient P's airways to tissue located at and around the target. More specifically, CT images acquired from previous CT scans are processed and assembled into a three-dimensional CT volume, which is then utilized to generate a three-dimensional model of patient P's airways. The three-dimensional model may be displayed on a display associated with the computer system 122, or in any other suitable fashion. Using the computer system 122, various views of the three-dimensional model or enhanced two-dimensional images generated from the three-dimensional model are presented. The enhanced two-dimensional images may possess some three-dimensional capabilities because they are generated from three-dimensional data. The three-dimensional model may be manipulated to facilitate identification of target on the three-dimensional model or two-dimensional images, and selection of a suitable pathway through patient P's airways to access tissue located at the target can be made. Once selected, the pathway plan, three-dimensional model, and images derived therefrom, can be saved and exported to a navigation system for use during the navigation phase or phases. One such planning software is the ILLUMISITE® planning suite currently sold by Medtronic PLC.

With respect to the navigation phase, a six degrees-of-freedom electromagnetic locating or tracking system 116, or other suitable system for determining location, is utilized for performing registration of the images and the pathway for navigation, although other configurations are also contemplated. The tracking system 116 includes the tracking module, the patient reference sensors 118, and the transmitter mat 120 (including the markers). The tracking system 116 is configured for use with a locatable guide 110 and particularly sensor 104. As described above, the locatable guide 110 and the sensor 104 are configured for insertion through the catheter 102 into patient P's airways (either with or without the bronchoscope 108) and are selectively lockable relative to one another via a locking mechanism.

The transmitter mat 120 is positioned beneath patient P. Transmitter mat 120 generates an electromagnetic field around at least a portion of the patient P within which the positions of the patient reference sensors 118 and the sensor 104 can be determined with use of a tracking system 116. A second electromagnetic sensor 126 may also be incorporated into the end of the catheter 102. The second electromagnetic sensor 126 may be a five degree-of-freedom sensor or a six degree-of-freedom sensor. One or more of the patient reference sensors 118 are attached to the chest of the patient P. The six degrees of freedom coordinates of the patient reference sensors 118 are sent to the computer system 122 (which includes the appropriate software) where they are used to calculate a patient coordinate frame of reference. Registration is generally performed to coordinate locations of the three-dimensional model and two-dimensional images from the planning phase, with the patient P's airways as observed through the bronchoscope 108, and allow for the navigation phase to be undertaken with precise knowledge of the location of the sensor 104, even in portions of the airway where the bronchoscope 108 cannot reach.

Registration of the patient P's location on the transmitter mat 120 may be performed by moving the sensor 104 through the airways of the patient P. More specifically, data pertaining to locations of the sensor 104, while the locatable guide 110 is moving through the airways, is recorded using the transmitter mat 120, the patient reference sensors 118, and the tracking system 116. A shape resulting from this location data is compared to an interior geometry of passages of the three-dimensional model generated in the planning phase, and a location correlation between the shape and the three-dimensional model based on the comparison is determined, e.g., utilizing the software on the computer system 122. In addition, the software identifies non-tissue space (e.g., air filled cavities) in the three-dimensional model. The software aligns, or registers, an image representing a location of the sensor 104 with the three-dimensional model and/or two-dimensional images generated from the three-dimension model, which are based on the recorded location data and an assumption that the locatable guide 110 remains located in non-tissue space in patient P's airways. Alternatively, a manual registration technique may be employed by navigating the bronchoscope 108 with the sensor 104 to pre-specified locations in the lungs of the patient P, and manually correlating the images from the bronchoscope to the model data of the three-dimensional model.

Though described herein with respect to EMN systems using EM sensors, the instant disclosure is not so limited and may be used in conjunction with flexible sensor, ultrasonic sensors, or without sensors. Additionally, the methods described herein may be used in conjunction with robotic systems such that robotic actuators drive the catheter 102 or bronchoscope 108 proximate the target.

Following registration of the patient P to the image data and pathway plan, a user interface is displayed in the navigation software which sets for the pathway that the clinician is to follow to reach the target. Once the catheter 102 has been successfully navigated proximate the target as depicted on the user interface, the locatable guide 110 may be unlocked from the catheter 102 and removed, leaving the catheter 102 in place as a guide channel for guiding medical devices including without limitation, optical systems, ultrasound probes, marker placement tools, biopsy tools, ablation tools (i.e., microwave ablation devices), laser probes, cryogenic probes, sensor probes, and aspirating needles to the target. A medical device may be then inserted through catheter 102 and navigated to the target or to a specific area adjacent to the target.

Prior to inserting the medical device through the catheter 102, a local registration process may be performed for each target to reduce the CT-to-body divergence. In a capture phase of the local registration process, a sequence of fluoroscopic images may be captured and acquired via the fluoroscopic imaging system 124, optionally by a user and according to directions displayed via the computer system 122. A fluoroscopic 3D reconstruction may be then generated via the computer system 122. The generation of the fluoroscopic 3D reconstruction is based on the sequence of fluoroscopic images and the projections of structure of markers incorporated with the transmitter mat 120 on the sequence of images. One or more slices of the 3D reconstruction may be then generated based on the pre-operative CT scan and via computer system 122. The one or more slices of the 3D reconstruction and the fluoroscopic 3D reconstruction may be then displayed to the user on a display via the computer system 122, optionally simultaneously. The slices of 3D reconstruction may be presented on the user interface in a scrollable format where the user is able to scroll through the slices in series.

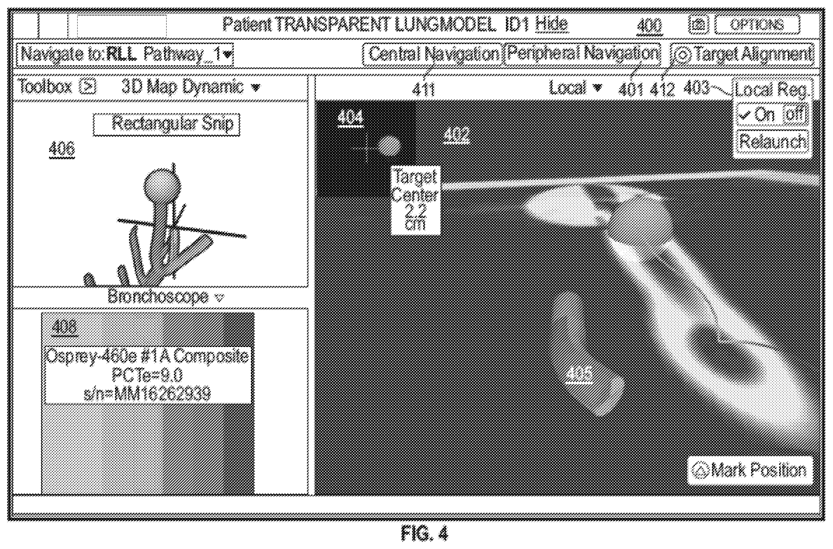

In a marking phase of the local registration process, the clinician may be directed to identify and mark the target while using the slices of the 3D reconstruction as a reference. The user may also be directed to identify and mark the navigation catheter tip in the sequence of fluoroscopic 2D images. An offset between the location of the target and the navigation catheter tip may be then determined or calculated via the computer system 122. The offset may be then utilized, via the computer system 122, to correct the location and/or orientation of the navigation catheter on the display (e.g., in the peripheral navigation screen which may be viewed by selecting the “Peripheral Navigation” tab 401 illustrated in FIG. 4) with respect to the target and/or correct the registration between the three-dimensional model and tracking system 116 in the area of the target and/or generate a local registration between the three-dimensional model and the fluoroscopic 3D reconstruction in the target area.

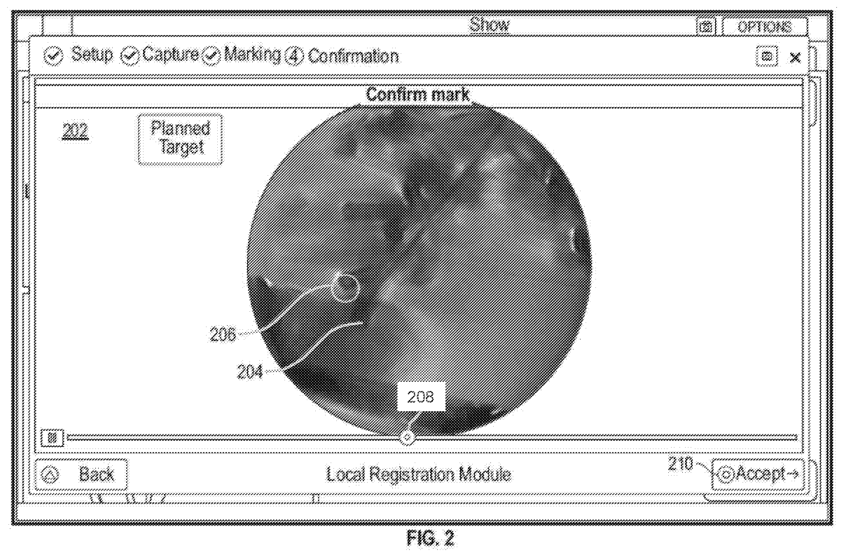

In a confirmation phase of the local registration process, a fluoroscopic 3D reconstruction is displayed in a confirmation screen 202, which is illustrated in FIG. 2. The confirmation screen 202 includes a slider 208 that may be selected and moved by the user to review a video loop of the fluoroscopic 3D reconstruction, which shows the marked target and navigation catheter tip from different perspectives. After confirming that there are marks on the target and navigation catheter tip throughout the video, the clinician may select the “Accept” button 210, at which point the local registration process ends and the position of the navigation catheter is updated. The clinician may then use the navigation views in, for example, the peripheral navigation screen illustrated in FIG. 4 to fine tune the alignment of the navigation catheter to the target before beginning an endoscopic procedure.

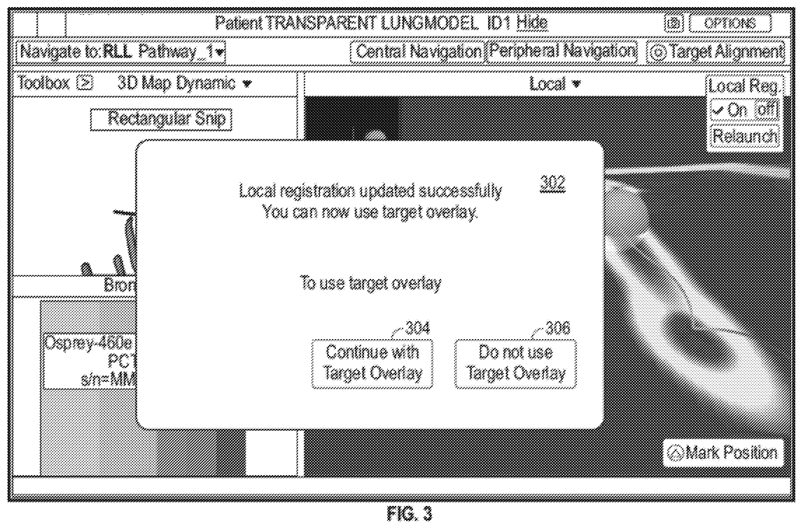

After the local registration process, the clinician or robot may insert a medical device in the catheter 102 and advance the medical device towards the target. While advancing the medical device towards the target, the clinician may view a user interface screen which includes: (a) a 3D medical device tip view of a 3D model of a target based on pre-operative CT scans, and (b) a live 2D fluoroscopic view on which a target marker corresponding to the 3D model of the target is overlaid. This user interface screen allows the clinician to not only see the medical device in real-time, but also allows the clinician to see whether the medical device is aligned with the target. The user interface screen may also provide a graphical indication of whether the medical device is aligned in three-dimensions with the target. For example, when the medical device is aligned in three-dimensions with the target, the user interface shows the target overlay in a first color, e.g., green. On the other hand, when the medical device is not aligned with the target in three dimensions, the user interface shows the target overlay in a second color different from the first color, e.g., orange or red.
FIG. 2 is a screen shot of a confirmation screen 202 of an example local registration user interface that appears during the confirmation phase of the local registration process. The confirmation screen 202 displays the navigation catheter tip mark 204 and the target mark 206, which were previously marked by the clinician during the marking phase of the local registration process. After the clinician selects the “Accept” button, the navigation user interface of FIG. 3 is displayed with a pop-up message 302. The pop-up message 302 may include buttons 304, 306, which enable the clinician to select whether to use the target overlay feature to guide navigation of a medical device, e.g., a biopsy tool, to the target. Specifically, the clinician may select button 304 to continue with the target overlay feature or the clinician may select button 306 to continue without using the target overlay feature.

When the clinician selects button 306, the peripheral navigation screen associated with the “Peripheral Navigation” tab 401 of the user interface 400 of FIG. 4, which was previously displayed prior to performing the local registration process, is redisplayed showing adjustments, if any, to the position and/or orientation of the navigation catheter tip 405 as a result of the location registration process. The peripheral navigation screen 401 includes a local CT view 402, a 3D navigation catheter tip view 404, a 3D map view 406, and a bronchoscope view 408. The peripheral navigation screen 401 also includes local registration user controls 403 enabling the user to apply local registration and/or relaunch local registration. The user interface 400 also includes a “Central Navigation” tab 411 and a “Target Alignment” tab 412, which may be individually selected to perform central navigation or target alignment, respectively.

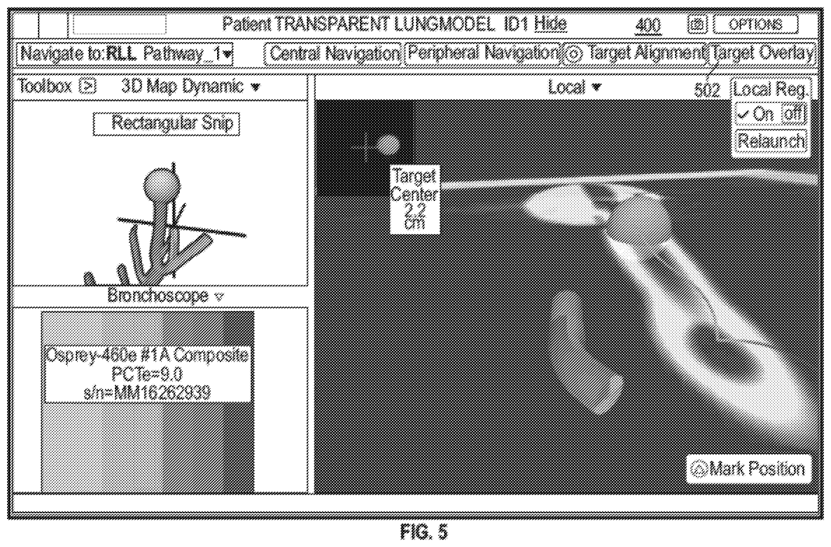

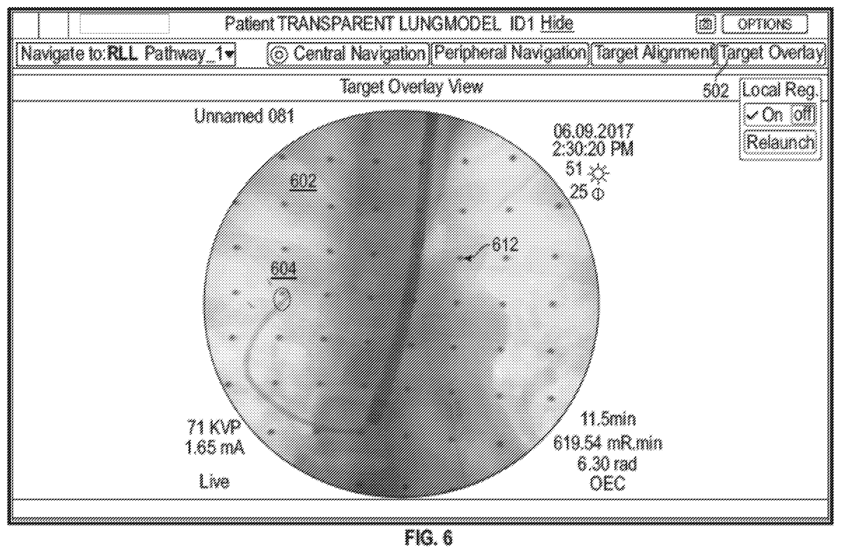

When the clinician selects a button of the local registration user controls 403, the user interface 400 displays the peripheral navigation screen and a “Target Overlay” tab 502 illustrated in FIG. 5. When the target overlay tab 502 is selected, a target overlay screen is displayed as shown in FIG. 6 which includes a live 2D fluoroscopic image or video 602 of the catheter 102 in the patient P.

FIG. 7 is a flowchart of an example of a method for visualizing a medical procedure on live fluoroscopic video, e.g., in the live fluoroscopic view. At block 702, local registration is performed. The fluoroscopic sweep for local registration may be taken through about 30-60 degrees to generate the 3D volumetric reconstruction. In aspects, the fluoroscopic sweep for local registration may be taken through a range of about 200 degrees.

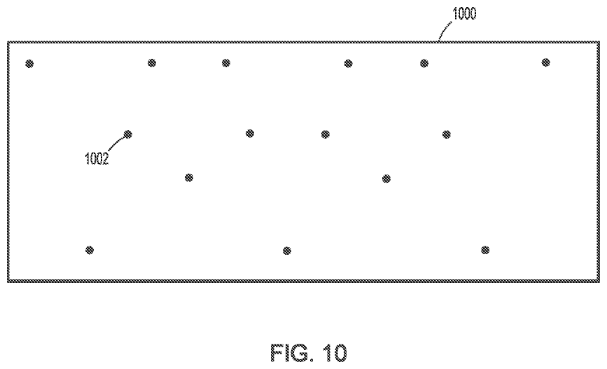

At block 704, a setup procedure is performed. The setup procedure may involve adjusting the C-arm fluoroscope such that the C-arm fluoroscope is properly aligned with the patient's body, and marking and confirming the marking of the locatable guide, e.g., a tip of the locatable, in a reference frame of the live fluoroscopic view. In other aspects, a 3D fluoroscope or a cone beam computed tomography (CBCT) imaging system may be used instead of the C-arm fluoroscope. The reference frame can be captured when the fluoroscopic imaging system 124, e.g., a fluoroscope, is in the AP position as well as at angles to the AP position without impeding the utility of the methods of this disclosure. The reference frame may include a projection of a structure of markers or fiducials, e.g., radiopaque markers or beads, for determining position information, e.g., the position of the fluoroscopic imaging system. The fiducial structure may be a non-periodic fiducial structure, which provides the ability to identify exact positions when evaluating a small region of the fiducial structure captured in the “Field of View” by the fluoroscope 124. FIG. 10 illustrates an example of a reference frame 1000 including a projection of a nonperiodic structure of radiopaque markers 1002, which provides more accurate position information than a periodic grid of radiopaque markers.

In the case where the grid is periodic, the setup procedure may be needed to improve C-arm source pose estimation from a single fluoroscopic image and to solve C-arm translations relative to the antenna. The setup procedure may include marking or determining a first position of a tip of a catheter in a reference frame of live fluoroscopic video; and determining a position of the fluoroscopic imaging system based on the first position of the tip of the catheter in the reference frame. The tip of the catheter may be marked manually or may be determined automatically using a segmentation algorithm or any other suitable algorithm or image recognition process for detecting the tip of the catheter. If the tip of the catheter is detected automatically, a reference frame may not be needed, and the setup procedure may occur automatically whenever movement of the fluoroscopic imaging system is detected. In the case where the grid is nonperiodic, the C-arm source pose may be estimated without the need for the setup procedure, e.g., without the need for the clinician or user to mark the tip of the catheter in the reference frame of the live fluoroscopic video. At block 706, the live fluoroscopic video is displayed in a user interface as illustrated herein. At block 708, the position of the target in the live fluoroscopic video is determined.

After the position of the target is determined in the live fluoroscopic video at block 708, a marking is displayed overlaid on the live fluoroscopic video at the determined position of the target at block 710. The method 700 may include panning a view of the live fluoroscopic video such that the target is at a center of the view of the live fluoroscopic video. Alternatively, the view may be automatically panned according to any other suitable automatic panning protocol that relies on the determined positions of the target and/or medical tool, e.g., catheter, in the fluoroscopic video. For example, the panning protocol may specify that the target be in a preset quadrant of the view of the live fluoroscopic video. At block 712, the computer system 122 determines whether there is a change in alignment of the catheter 102, e.g., an extended working channel, with the target. If there is a change in alignment of the catheter 102 with the target, the appearance of the marker is updated at block 714. The update to the appearance of the marker may include changing the color of the marker. In the case where the marker is a circle or ellipse, the update to the appearance of the marker may include changing the dash type of the line forming the circle or ellipse.

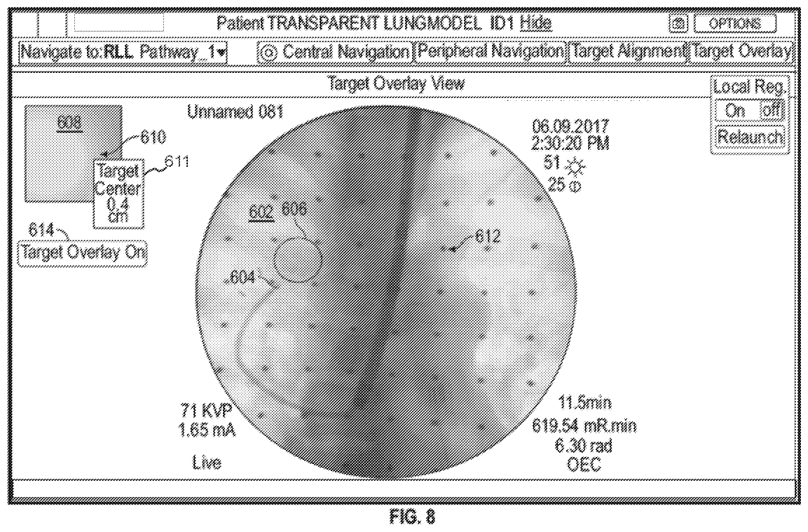

In the example of FIG. 8, the tip 604 of the catheter 102 is shown as aligned with the target marker 606, which is displayed in the live 2D fluoroscopic image 602. In addition, a medical device tip view 608 depicts the view as if a camera were located at the tip 604 of the catheter 102. The medical device tip view 608 presents a three-dimensional (3D) representation of the target 610. If the tip 604 of the catheter 102 is nearly aligned with the target, the target marker 606 may be displayed in a first color (e.g., green) and overlaid on the live 2D fluoroscopic image 602. If, on the other hand, the tip 604 of the catheter 102 were not aligned with the target (for example, as shown in the 3D navigation catheter tip view 404 of FIG. 4 in which the spherical target is disposed to the right of the center of the 3D navigation catheter tip view 404), the target marker 606 may be displayed in a different color, e.g., orange or red. Similarly, in the medical device tip view 608 where the tip 604 of the catheter 102 is not aligned with the target, the 3D representation of the target 610 will appear offset in the image, and only a portion or none of it may be visible in that view. In addition, the color may also change as described above depending on the severity of the misalignment of the tip 604 of the catheter 102 and the target The medical device tip view 608 may also include a text box 611, which displays text indicating a distance between the tip 604 of the catheter 102 and the center of target. In aspects, the computer system 122 may calculate the distance by aligning or finding the correspondence between the 3D model of the luminal network, which may be based on a CT scan and which includes the target, and the live fluoroscopic view, and measuring the distance between the tip 604 and the center of the 3D model of the target using, for example, image processing. In finding the correspondence between the 3D model and the live fluoroscopic view, the fluoroscopic 3D reconstruction generated and marked in the local registration process may be used. In some aspects, the distance is measured from a center of or an outside edge of the target. The target overlay screen further includes a target overlay toggle button 614, which, when selected, toggles between displaying the target marker 606 as shown in FIG. 8 and not displaying the target marker 606.

In the example of FIG. 8, the tip 604 of the catheter 102 is shown as aligned with the target marker 606, which is displayed in the live 2D fluoroscopic image 602. In addition, a medical device tip view 608 depicts the view as if a camera were located at the tip 604 of the catheter 102. The medical device tip view 608 presents a three-dimensional representation of the target 610. If the tip 604 of the catheter 102 is nearly aligned with the target, the target marker 606 may be displayed in a first color (e.g., green) and overlaid on the live 2D fluoroscopic image 602. If, on the other hand, the tip 604 of the catheter 102 were not aligned with the target (for example, as shown in the 3D navigation catheter tip view 404 of FIG. 4 in which the spherical target is disposed to the right of the center of the 3D navigation catheter tip view 404), the target marker 606 may be displayed in a different color, e.g., orange or red. Similarly, in the medical device tip view 608 where the tip 604 of the catheter 102 is not aligned with the target, the 3D representation of the target 610 will appear offset in the image, and only a portion or none of it may be visible in that view. In addition, the color may also change as described above depending on the severity of the misalignment of the tip 604 of the catheter 102 and the target

The medical device tip view 608 may also include a text box 611, which displays text indicating a distance between the tip 604 of the catheter 102 and the center of target. In aspects, the computer system 122 may calculate the distance by aligning or finding the correspondence between the 3D model of the luminal network, which may be based on a CT scan and which includes the target, and the live fluoroscopic view, and measuring the distance between the tip 604 and the center of the 3D model of the target using, for example, image processing. In finding the correspondence between the 3D model and the live fluoroscopic view, the fluoroscopic 3D reconstruction generated and marked in the local registration process may be used. In some aspects, the distance is measured from a center of or an outside edge of the target. The target overlay screen further includes a target overlay toggle button 614, which, when selected, toggles between displaying the target marker 606 as shown in FIG. 8 and not displaying the target marker 606.

Turning again to FIG. 7, if there is no change in alignment of the catheter 102 with the target, the computer system 122 determines whether movement of the patient's body is detected at block 716. Movement of the patient's body may be detected by one or more patient reference sensors disposed on the patient's body, e.g., one or more of the patient reference sensors 118 depicted in FIG. 1. If movement of the patient's body is detected at block 716, the method 700 returns to block 708 to determine the new position of the target in the live fluoroscopic video. The new position of the target in the live fluoroscopic video may be determined based on position information provided by the one or more patient reference sensors.

If movement of the patient's body is not detected at block 716, the method 700 determines whether there is movement of the C-arm fluoroscope or change in the optic magnification of the camera or image intensifier of the C-arm fluoroscope at block 718. Movement of the C-arm fluoroscope may include translation and/or rotation of the C-arm fluoroscope. C-arm translation and/or rotation may be determined based on movement of the locatable guide antenna using the periodic grid of radiopaque markers 612 or a nonperiodic grid of radiopaque markers (e.g., the nonperiodic grid illustrated in FIG. 10) adjacent the transmitter mat 120. If there is movement of the C-arm fluoroscope or change in the optic magnification of the camera or image intensifier of the C-arm fluoroscope at block 718, the method 700 returns to block 704 to reperform the setup procedure or to re-estimate the position of the fluoroscopic imaging system based on the nonperiodic grid. Otherwise, the method 700 returns to block 710 to display the marking overlaid on the live fluoroscopic video at the position of the target determined at block 708.

In some aspects, the computer system 122 may monitor for both the movement of the fluoroscopic imaging system and the patient in parallel, and may update the position of the target overlaid on the live fluoroscopic video based on the movement of the patient.

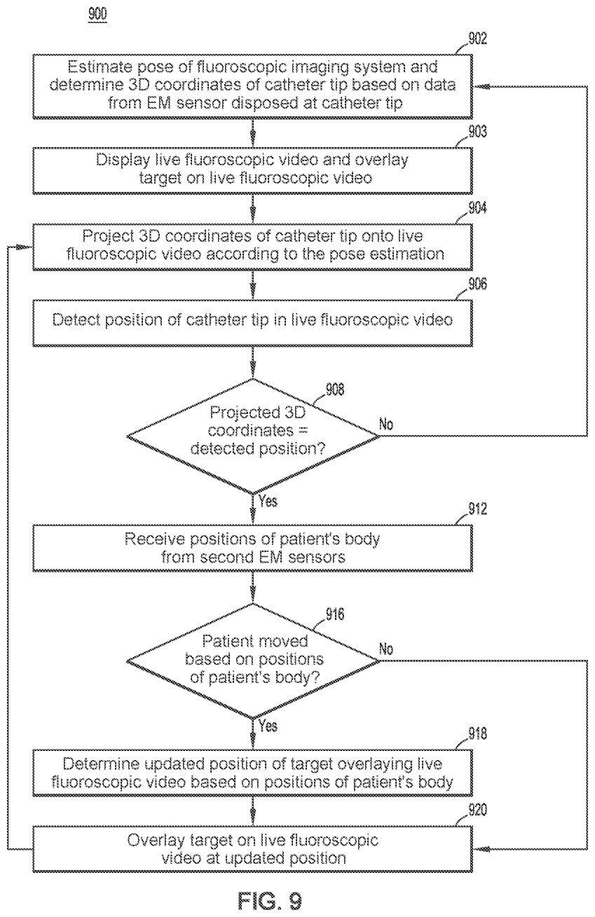

In some aspects, the computer system 122 may detect movement of the fluoroscopic imaging system without the use of a reference frame. FIG. 9 illustrates a method 900 for determining whether the fluoroscopic imaging system and/or the patient's body has moved, in which case the position of the target overlaid on the live fluoroscopic video is updated based on the movement of the patient's body. In some aspects, movement of the fluoroscopic imaging system is determined without using a reference frame.

In one aspect, the method 900 includes continuously projecting the 3D coordinates of the catheter tip EM sensor onto the live fluoroscopic video with the solved position of the fluoroscopic imaging system from the setup phase. Accordingly, at block 902, which may be performed in the setup phase, a pose of the fluoroscopic imaging system is estimated and 3D coordinates of the tip of the catheter, e.g., the locatable guide, EWC, or biopsy tool, is determined based on data from a first electromagnetic (EM) sensor disposed at the tip of the catheter. At block 903, live fluoroscopic video is displayed, and a target or representation of the target is overlayed on the live fluoroscopic video. At block 904, 3D coordinates of the catheter tip are projected onto live fluoroscopic video according to the pose estimation. At block 906, a position of the tip of the catheter is detected in the live fluoroscopic video. In one example, to detect the position of the catheter tip, image analysis may be employed to identify pixels in the live 2D fluoroscopic image 602 that have a Hounsfield unit value greater than a threshold, above which the Hounsfield unit values correspond to the radiopaque catheter. The last connected pixel of the pixels making up the catheter 102 may be identified as the tip 604 of the catheter 102. Other processes may also be used for detecting the tip 604 of the catheter 102 in the live fluoroscopic video without departing from the scope of the disclosure. For example, either an automatic computer vision algorithm or a user interface enabling a user to mark a catheter tip manually may be used to detect if the EM sensor (the catheter tip) is visible in the fluoroscopic video.

At block 908, the method 900 determines whether the fluoroscopic imaging system 124 moved based on whether the projected 3D coordinates match or closely match the position of the catheter tip detected in the live fluoroscopy video. If the method 900 determines that the fluoroscope moved at block 908, the setup procedure including block 902 is repeated. The new position of the fluoroscopic imaging system may be determined based at least partially on determining a translation. Because of the periodic grid markers, there is a discrete number of possible translations that could have occurred. The computer system 122 may evaluate the possible translations and select, from the possible translations, a translation for which the projection of the EM sensor on the catheter tip matches a catheter tip detection in the live fluoroscopic video. If, on the other hand, the method 900 determines that the fluoroscope did not move at block 908, the method 900 proceeds to block 912.

At block 912, the computer system 122 receives positions of the patient's body from one or more second EM sensors disposed on the patient's body. At block 916, the method 900 determines whether the patient's body moved based on the positions of the patient's body. If the method 900 determines that the patient's body moved based on the positions of the patient's body at block 916, an updated position of the target overlaying the live fluoroscopic video is determined based on the positions of the patient's body at block 918, the target is overlaid on the live fluoroscopic video at the updated position of the target at block 920, and the method 900 returns to block 904. If the method 900 determines that the patient's body moved, the method 900 returns to block 904.

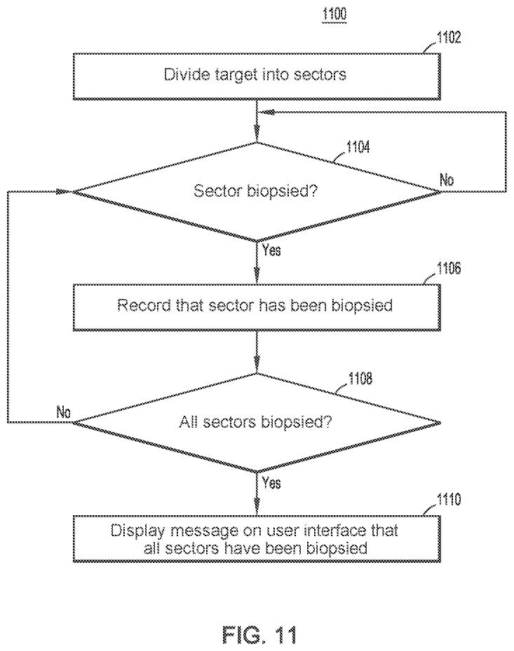

FIG. 11 depicts a method 1100 for tracking a biopsy procedure. At block 1102, the target is divided into sectors. The target may be divided into sectors by determining the shape and volume of the target from preoperative images, e.g., computed tomography (CT) images and dividing the target into sectors based on the shape and volume of the target. The method 1100 may include displaying the target and enabling the clinician to mark in the volume the sectors to be biopsied. The method 1100 then determines, either automatically (e.g., by an image processing algorithm) or manually (e.g., by a user marking a biopsied sector through a user interface), whether a sector has been biopsied, at block 1104, and records that the sector has been biopsied, at block 1106. At block 1108, the method 1100 determines whether all sectors have been biopsied. If all sectors have been biopsied, the method 1100 returns to block 1104 to determine whether another target sector has been biopsied. Otherwise, the method 1100 displays a message on the user interface that all sectors have been biopsied at block 1110.

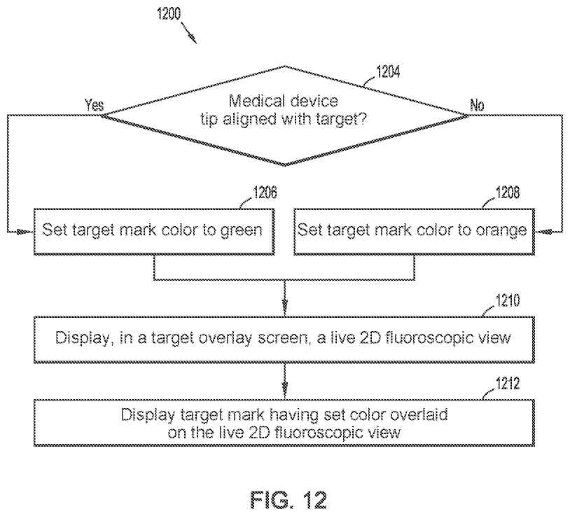

FIG. 12 is a flow diagram of an example of a method 1200 that may implement blocks 712 and 714 of FIG. 7 in order to visualize the navigation of the medical device tip towards a target after the medical device tip is brought into the vicinity of the target. The computer system 122 determines whether the medical device tip is aligned with the target at block 1204. The computer system 122 may determine whether the medical device tip is aligned with the target by aligning or finding the correspondence between the 3D model of the luminal network, which may be based on a CT scan and which includes the target, and the live fluoroscopic view; and determine whether the medical device tip is aligned with the 3D model of the target based on the determined alignment or correspondence and applying, for example, image processing. In aligning or finding the correspondence between the 3D model and the live fluoroscopic view, the fluoroscopic 3D reconstruction generated and marked in the local registration process may be used.

When the computer system 122 determines that the medical device tip is aligned with the target, the computer system 122 sets the target mark color to green at block 1406; otherwise, the computer system 122 sets the target mark color to orange at block 1408. At block 1210, the computer system 122 displays, in a target overlay screen, a live 2D fluoroscopic view, which at least shows the medical device. At block 1212, the computer system 122 displays a target mark having the set color overlaid on the live 2D fluoroscopic view. At block 1214, the computer system 122 displays, in the same target overlay screen, a 3D virtual target, which corresponds to the target mark, from the perspective of the medical device tip. Blocks 1204-1212 may be repeated until the medical device tip is placed at the center of the target or until the biopsy or other treatment is completed. This final navigation allows the user to use fluoroscopic navigation techniques to obtain live images with the target marked on the live images, which enables the user to see how well the medical device tip is aligned with the target to ensure that the medical device tip reaches the target to take a sample of the target or perform treatment on the target.

In a further aspect of the disclosure, following the local registration (e.g., block 702 of FIG. 7) the computer system 122 may undertake an image analysis of the fluoroscopic 3D reconstruction to determine an angle for placement of the fluoroscopic imaging system 124 to optimally engage in the target overlay tab. In this aspect of the disclosure, following the identification of the tip of the catheter 102 in two slices of the fluoroscopic 3D reconstruction, and identification of the target in the fluoroscopic 3D reconstruction and determining the relative position of the tip of the catheter 102 and the target in the fluoroscopic 3D reconstruction, the computer system 122 performs an image analysis of the 3D reconstruction to determine a slice of the 3D reconstruction at which the catheter and the target visible. This may be the slice where both the target and the catheter 102 are most visible or most visible beyond some minimum threshold. Those of skill in the art will appreciate that there will be slices in which one or the other (or both) of the catheter or target are not visible and those images will likely be ignored by the computer system 122 when performing this analysis.

After analyzing the remaining slices of the fluoroscopic 3D reconstruction, one of the slices is identified as most clearly depicting both the catheter 102 and the target. Once the slice of the 3D reconstruction is determined, the position (e.g., angle to the patient P or operating table 112) of the fluoroscopic imaging system 124 where a corresponding 2D fluoroscopic image, such as the live 2D fluoroscopic image 602, can be captured. This position of the fluoroscopic imaging system 124 can be presented to the clinician on a user interface prior to engaging the “Target Overlay” tab 502, so that they can manually move the fluoroscopic imaging system 124 to that position. Alternatively, the fluoroscopic imaging system 124 may receive an indication of the position determined by the computer system 122 and automatically drive the fluoroscopic imaging system 124 to that position such that upon selecting the “Target Overlay” tab 502 the live 2D fluoroscopic image 602 is acquired at this pre-determined optimum position for viewing the catheter 102 and target.

Another aspect of the disclosure is the enablement of the use of zoom features which may be built into the fluoroscopic imaging system 124. As depicted in FIG. 6, the live 2D fluoroscopic image 602 includes a plurality of radiopaque markers 612. These radiopaque markers 612 may be placed on or embedded in the transmitter mat 120. The distances between the radiopaque markers are fixed and known by the computer system 122. Because the distances between the radiopaque markers 612 is known, if the distance between any of the markers exceeds the known distances the computer system 122 can determine that the zoom features of the fluoroscopic imaging system 124 are engaged. The exact amount of zoom that has been engaged can be determined by comparing the spacing of radiopaque markers 612 in the live 2D fluoroscopic image 602 to the known spacing of the radiopaque markers 612 in the transmitter mat 120. Once the amount of zoom is determined, the computer system 122 can calculate an offset in the relative position of the tip 604 of the catheter 102 and the target such that the target marker 606 can be accurately displayed in the live 2D fluoroscopic image 602 despite the change in zoom from when the local registration process was undertaken.

Reference is now made to FIG. 13, which is a schematic diagram of a system 1300 configured for use with the methods of the disclosure including the method of FIG. 12. System 1300 may include a workstation 1301, and optionally a fluoroscopic imaging system or fluoroscope 1315. In some aspects, the workstation 1301 may be coupled with fluoroscope 1315, directly or indirectly, e.g., by wireless communication. Workstation 1301 may include a memory 1302, a processor 1304, a display 1306 and an input device 1310. The processor 1304 may include one or more hardware processors. The workstation 1301 may optionally include an output module 1312 and a network interface 1008. The memory 1302 may store an application 1318 and image data 1314. The application 1318 may include instructions executable by the processor 1304 for executing the methods of the disclosure including the methods of FIGS. 7, 9, 11, and 12.

The application 1318 may further include a user interface 1316. The image data 1314 may include the CT scans, the generated fluoroscopic 3D reconstructions of the target area and/or any other fluoroscopic image data and/or the generated one or more slices of the 3D reconstruction. The processor 1304 may be coupled with the memory 1302, the display 1306, the input device 1310, the output module 1312, the network interface 1308, and the fluoroscope 1315. The workstation 1301 may be a stationary computer system, such as a personal computer, or a portable computer system such as a tablet computer. The workstation 1301 may embed multiple computer systems.

The memory 1302 may include any non-transitory computer-readable storage media for storing data and/or software including instructions that are executable by the processor 1304 and which control the operation of workstation 1301 and, in some aspects, may also control the operation of the fluoroscope 1315. The fluoroscope 1315 may be used to capture a sequence of fluoroscopic images based on which the fluoroscopic 3D reconstruction is generated and to capture a live 2D fluoroscopic view according to this disclosure. In one aspect, the memory 1302 may include one or more storage devices such as solid-state storage devices, e.g., flash memory chips. Alternatively, or in addition to the one or more solid-state storage devices, the memory 1302 may include one or more mass storage devices connected to the processor 1304 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 1304. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 1301.

The application 1318 may, when executed by the processor 1304, cause the display 1306 to present the user interface 1316. The user interface 1316 may be configured to present to the user a single screen including a three-dimensional (3D) view of a 3D model of a target from the perspective of a tip of a medical device, a live two-dimensional (2D) fluoroscopic view showing the medical device, and a target mark, which corresponds to the 3D model of the target, overlaid on the live 2D fluoroscopic view, as shown, for example, in FIG. 8. The user interface 1316 may be further configured to display the target mark in different colors depending on whether the medical device tip is aligned with the target in three dimensions.

The network interface 1308 may be configured to connect to a network such as a local area network (LAN) consisting of a wired network and/or a wireless network, a wide area network (WAN), a wireless mobile network, a Bluetooth network, and/or the Internet. The network interface 1308 may be used to connect between the workstation 1301 and the fluoroscope 1315. The network interface 1308 may be also used to receive the image data 1314. The input device 1310 may be any device by which a user may interact with the workstation 1301, such as, for example, a mouse, keyboard, foot pedal, touch screen, and/or voice interface. The output module 1312 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

While detailed aspects are disclosed herein, the disclosed aspects are merely examples of the disclosure, which may be embodied in various forms and aspects. For example, aspects of an electromagnetic navigation system, which incorporates the target overlay systems and methods, are disclosed herein; however, the target overlay systems and methods may be applied to other navigation or tracking systems or methods known to those skilled in the art. Therefore, specific structural and functional details disclosed herein are not to be interpreted as limiting, but merely as a basis for the claims and as a representative basis for teaching one skilled in the art to variously employ the disclosure in virtually any appropriately detailed structure.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of aspects. Those skilled in the art will envision other modifications within the scope and spirit of the claims appended hereto.

Aspects of the disclosure may be further described by reference to the following numbered paragraphs:
1. A method comprising:
   performing a setup procedure including:
      determining a first position of a tip of a catheter in a reference frame of a live fluoroscopic video; and
      determining a position of a fluoroscopic imaging system based on the first position of the tip of the catheter in the reference frame;
   receiving live fluoroscopic video from a fluoroscopic imaging system;
   displaying the live fluoroscopic video;
   projecting three-dimensional (3D) coordinates from an electromagnetic (EM) sensor disposed at the tip of the catheter onto the live fluoroscopic video based on the position of the fluoroscopic imaging system, yielding projected 3D coordinates;
   overlaying a target on the live fluoroscopic video;
   determining that a second position of the tip of the catheter in the live fluoroscopic video is not at or near the projected 3D coordinates;
   in response to determining that the second position of the tip of the catheter in the live fluoroscopic video is not at or near the projected 3D coordinates, determining that the fluoroscopic imaging system has moved and repeating performing the setup procedure;
   receiving a position of a patient from one or more second EM sensors disposed on the patient;
   determining that the patient has moved based on the position of the patient;
   in response to determining that the patient has moved, determining an updated position of the target; and
   overlaying the target on the live fluoroscopic video at the updated position.
2. The method of paragraph 1, further comprising:
   determining that the catheter is not aligned with the target; and
   in response to determining that the catheter is not aligned with the target, updating an appearance of the target overlaying the live fluoroscopic video.
3. The method of paragraph 2, wherein updating the appearance of the target includes changing a color of the target, highlighting the target, or applying a line pattern to the target.
4. The method of paragraph 1, further comprising panning a view of the live fluoroscopic video such that the target is at a center of the view of the live fluoroscopic video.
5. The method of paragraph 1, wherein determining that the patient has moved includes:
   determining a reference position of the one or more second EM sensors; and
   determining that a distance between the position of the patient and the reference position is greater than a threshold distance.
6. The method of paragraph 2, further comprising:
   determining possible translations of the fluoroscopic imaging system based on a periodic grid appearing in the live fluoroscopic video;
   selecting, from the possible translations, a translation for which projected 3D coordinates most closely matches the second position of the tip of the catheter in the live fluoroscopic video, yielding a selected translation; and
   determining the position of the fluoroscopic imaging system based on the first position of the tip of the catheter in the reference frame and the selected translation.
7. The method of paragraph 1, further comprising:
   tracking portions of the target that have been biopsied; and
   displaying the portions of the target that have been biopsied.
8. The method of paragraph 1, wherein the fluoroscopic imaging system is a 3D fluoroscope.
9. A system for guiding navigation of a biopsy tool in a patient, the system comprising:
   an electromagnetic (EM) field generator configured to generate an electromagnetic field;
   a first EM sensor disposed at a tip of a catheter;
   one or more second EM sensor disposed on the patient;
   a display;
   a processor; and
   a memory having stored thereon instructions, wherein when the instructions are executed by the processor, the processor:
      performs a setup procedure including:
         determining a first position of a tip of a catheter in a reference frame of a live fluoroscopic video; and
         determining a position of a fluoroscopic imaging system based on the first position of the tip of the catheter in the reference frame;
      receives live fluoroscopic video from a fluoroscopic imaging system;
      displays the live fluoroscopic video;
      projects three-dimensional (3D) coordinates from an electromagnetic (EM) sensor disposed at the tip of the catheter onto the live fluoroscopic video based on the position of the fluoroscopic imaging system, yielding projected 3D coordinates;
      overlays a target on the live fluoroscopic video;
      determines that a second position of the tip of the catheter in the live fluoroscopic video is not at or near the projected 3D coordinates;
      in response to determining that the second position of the tip of the catheter in the live fluoroscopic video is not at or near the projected 3D coordinates, determines that the fluoroscopic imaging system has moved and repeats performing the setup procedure;
      receives a position of a patient from the one or more second EM sensors disposed on the patient;
      determines that the patient has moved based on the position of the patient;
      in response to determining that the fluoroscopic imaging system has moved or the patient has moved, determines an updated position of the target; and
      overlays the target on the live fluoroscopic video at the updated position.
10. The system of paragraph 9, wherein, when the instructions are executed by the processor, the processor further calculates a patient coordinate frame of reference based on the EM field sensed by the EM sensors.
11. The system of paragraph 9, wherein, when the instructions are executed by the processor, the processor further:
   determines that the catheter is not aligned with the target; and
   in response to determining that the catheter is not aligned with the target, updates an appearance of the target overlaying the live fluoroscopic video.
12. The system of paragraph 11, wherein updating the appearance of the target overlaying the live fluoroscopic video includes changing a color of the target, highlighting the target, or applying a line pattern to the target.
13. The system of paragraph 9, wherein, when the instructions are executed by the processor, the processor further pans a view of the live fluoroscopic video such that the target is at a center of the view of the live fluoroscopic video.
14. The system of paragraph 9, wherein, when the instructions are executed by the processor, the processor further:
   determines possible translations of the fluoroscopic imaging system based on a periodic grid appearing in the live fluoroscopic video;
   selects, from the possible translations, a translation for which projected 3D coordinates most closely matches the second position of the tip of the catheter in the live fluoroscopic video, yielding a selected translation; and
   determines the position of the fluoroscopic imaging system based on the first position of the tip of the catheter in the reference frame and the selected translation.
15. The system of paragraph 9, wherein, when the instructions are executed by the processor, the processor further:
   tracks portions of the target that have been biopsied; and displays the portions of the target that have been biopsied.
16. The system of paragraph 9, wherein the fluoroscopic imaging system is a 3D fluoroscope.
17. A system comprising:
   an electromagnetic (EM) field generator configured to generate an electromagnetic field;
   one or more EM sensors disposed on a body of a patient;
   a display;
   a processor coupled to the display; and
   a memory coupled to the processor and having stored thereon instructions, which when executed by the processor, cause the processor to:
      display, on the display, a screen including a three-dimensional (3D) view of a 3D model of a target from a perspective of a tip of a medical device;
      display, in the screen, a live two-dimensional (2D) fluoroscopic view showing the medical device;
      overlay a target mark, which corresponds to the 3D model of the target, on the live 2D fluoroscopic view;
      determine that the body of the patient has moved based on one or more signals received from the one or more EM sensors; and
      in response to determining that the body of the patient has moved, update a 2D position of the target mark overlaying the live 2D fluoroscopic view.
18. The system of paragraph 17, wherein determining that the body of the patient has moved includes determining that an EM sensor of the one or more EM sensors disposed on the patient has moved greater than a threshold amount.
19. The system of paragraph 17, wherein determining that the body of the patient has moved includes determining that a combination of two or more of the EM sensors disposed on the patient have moved greater than a threshold amount in a particular direction.
20. A method comprising:
   detecting a nonperiodic grid of markers in at least one fluoroscopic image captured by a fluoroscopic imaging system, yielding a detected nonperiodic grid of markers;
   determining a position of the fluoroscopic imaging system based on the detected nonperiodic grid of markers;
   displaying live fluoroscopic video from the fluoroscopic imaging system;
   projecting three-dimensional (3D) coordinates from an electromagnetic (EM) sensor disposed at a tip of a catheter onto the live fluoroscopic video based on the position of the fluoroscopic imaging system, yielding projected 3D coordinates;
   overlaying a target on the live fluoroscopic video;
   determining that a second position of the tip of the catheter in the live fluoroscopic video is not at or near the projected 3D coordinates;
   in response to determining that the second position of the tip of the catheter in the live fluoroscopic video is not at or near the projected 3D coordinates, determining that the fluoroscopic imaging system has moved; and
   in response to determining that the fluoroscopic imaging system has moved, repeating the detecting the nonperiodic grid of markers and determining the position of the fluoroscopic imaging system.

What is claimed is:

1. A method comprising:
performing a setup procedure including:
determining a first position of a tip of a catheter in a reference frame of a live fluoroscopic video; and
determining a position of a fluoroscopic imaging system based on the first position of the tip of the catheter in the reference frame;
receiving live fluoroscopic video from a fluoroscopic imaging system;
displaying the live fluoroscopic video;
projecting three-dimensional (3D) coordinates from an electromagnetic (EM) sensor disposed at the tip of the catheter onto the live fluoroscopic video based on the position of the fluoroscopic imaging system, yielding projected 3D coordinates;
overlaying a target on the live fluoroscopic video;
determining that a second position of the tip of the catheter in the live fluoroscopic video is not at or near the projected 3D coordinates;
in response to determining that the second position of the tip of the catheter in the live fluoroscopic video is not at or near the projected 3D coordinates, determining that the fluoroscopic imaging system has moved and repeating performing the setup procedure;
receiving a position of a patient from one or more second EM sensors disposed on the patient;
determining that the patient has moved based on the position of the patient;
in response to determining that the patient has moved, determining an updated position of the target; and
overlaying the target on the live fluoroscopic video at the updated position.

2. The method of claim 1, further comprising:
determining that the catheter is not aligned with the target; and
in response to determining that the catheter is not aligned with the target, updating an appearance of the target overlaying the live fluoroscopic video.

3. The method of claim 2, wherein updating the appearance of the target includes changing a color of the target, highlighting the target, or applying a line pattern to the target.

4. The method of claim 1, further comprising panning a view of the live fluoroscopic video such that the target is at a center of the view of the live fluoroscopic video.

5. The method of claim 1, wherein determining that the patient has moved includes:
determining a reference position of the one or more second EM sensors; and
determining that a distance between the position of the patient and the reference position is greater than a threshold distance.

6. The method of claim 2, further comprising:
determining possible translations of the fluoroscopic imaging system based on a periodic grid appearing in the live fluoroscopic video;
selecting, from the possible translations, a translation for which projected 3D coordinates most closely matches the second position of the tip of the catheter in the live fluoroscopic video, yielding a selected translation; and
determining the position of the fluoroscopic imaging system based on the first position of the tip of the catheter in the reference frame and the selected translation.

7. The method of claim 1, further comprising:
tracking portions of the target that have been biopsied; and
displaying the portions of the target that have been biopsied.

8. The method of claim 1, wherein the fluoroscopic imaging system is a 3D fluoroscope.

9. A system for guiding navigation of a biopsy tool in a patient, the system comprising:
an electromagnetic (EM) field generator configured to generate an electromagnetic field;
a first EM sensor disposed at a tip of a catheter;
one or more second EM sensor disposed on the patient;
a display;
a processor; and
a memory having stored thereon instructions, wherein when the instructions are executed by the processor, the processor:
performs a setup procedure including:
determining a first position of a tip of a catheter in a reference frame of a live fluoroscopic video; and
determining a position of a fluoroscopic imaging system based on the first position of the tip of the catheter in the reference frame;
receives live fluoroscopic video from a fluoroscopic imaging system;
displays the live fluoroscopic video;
projects three-dimensional (3D) coordinates from an electromagnetic (EM) sensor disposed at the tip of the catheter onto the live fluoroscopic video based on the position of the fluoroscopic imaging system, yielding projected 3D coordinates;
overlays a target on the live fluoroscopic video;
determines that a second position of the tip of the catheter in the live fluoroscopic video is not at or near the projected 3D coordinates;
in response to determining that the second position of the tip of the catheter in the live fluoroscopic video is not at or near the projected 3D coordinates, determines that the fluoroscopic imaging system has moved and repeats performing the setup procedure;
receives a position of a patient from the one or more second EM sensors disposed on the patient;
determines that the patient has moved based on the position of the patient;
in response to determining that the fluoroscopic imaging system has moved or the patient has moved, determines an updated position of the target; and
overlays the target on the live fluoroscopic video at the updated position.

10. The system of claim 9, wherein, when the instructions are executed by the processor, the processor further calculates a patient coordinate frame of reference based on the EM field sensed by the EM sensors.

11. The system of claim 9, wherein, when the instructions are executed by the processor, the processor further:
determines that the catheter is not aligned with the target; and
in response to determining that the catheter is not aligned with the target, updates an appearance of the target overlaying the live fluoroscopic video.

12. The system of claim 11, wherein updating the appearance of the target overlaying the live fluoroscopic video includes changing a color of the target, highlighting the target, or applying a line pattern to the target.

13. The system of claim 9, wherein, when the instructions are executed by the processor, the processor further pans a view of the live fluoroscopic video such that the target is at a center of the view of the live fluoroscopic video.

14. The system of claim 9, wherein, when the instructions are executed by the processor, the processor further:
determines possible translations of the fluoroscopic imaging system based on a periodic grid appearing in the live fluoroscopic video;
selects, from the possible translations, a translation for which projected 3D coordinates most closely matches the second position of the tip of the catheter in the live fluoroscopic video, yielding a selected translation; and
determines the position of the fluoroscopic imaging system based on the first position of the tip of the catheter in the reference frame and the selected translation.

15. The system of claim 9, wherein, when the instructions are executed by the processor, the processor further:
tracks portions of the target that have been biopsied; and
displays the portions of the target that have been biopsied.

16. The system of claim 9, wherein the fluoroscopic imaging system is a 3D fluoroscope.

17. A system comprising:
an electromagnetic (EM) field generator configured to generate an electromagnetic field;
one or more EM sensors disposed on a body of a patient;
a display;
a processor coupled to the display; and
a memory coupled to the processor and having stored thereon instructions, which when executed by the processor, cause the processor to:
display, on the display, a screen including a three-dimensional (3D) view of a 3D model of a target from a perspective of a tip of a medical device;
display, in the screen, a live two-dimensional (2D) fluoroscopic view showing the medical device;
overlay a target mark, which corresponds to the 3D model of the target, on the live 2D fluoroscopic view;
determine that the body of the patient has moved based on one or more signals received from the one or more EM sensors; and
in response to determining that the body of the patient has moved, update a 2D position of the target mark overlaying the live 2D fluoroscopic view.

18. The system of claim 17, wherein determining that the body of the patient has moved includes determining that an EM sensor of the one or more EM sensors disposed on the patient has moved greater than a threshold amount.

19. The system of claim 17, wherein determining that the body of the patient has moved includes determining that a combination of two or more of the EM sensors disposed on the patient have moved greater than a threshold amount in a particular direction.

20. A method comprising:
detecting a nonperiodic grid of markers in at least one fluoroscopic image captured by a fluoroscopic imaging system, yielding a detected nonperiodic grid of markers;
determining a position of the fluoroscopic imaging system based on the detected nonperiodic grid of markers;
displaying live fluoroscopic video from the fluoroscopic imaging system;
projecting three-dimensional (3D) coordinates from an electromagnetic (EM) sensor disposed at a tip of a catheter onto the live fluoroscopic video based on the position of the fluoroscopic imaging system, yielding projected 3D coordinates;
overlaying a target on the live fluoroscopic video;
determining that a second position of the tip of the catheter in the live fluoroscopic video is not at or near the projected 3D coordinates;
in response to determining that the second position of the tip of the catheter in the live fluoroscopic video is not at or near the projected 3D coordinates, determining that the fluoroscopic imaging system has moved; and
in response to determining that the fluoroscopic imaging system has moved, repeating the detecting the nonperiodic grid of markers and determining the position of the fluoroscopic imaging system.
